(19) United States
(12) Patent Application Publication
(10) Pub. No.: US 2003/0130628 A1
(43) Pub. Date: Jul. 10, 2003

(54) Method and apparatus for the treatment of scar tissue and wrinkles

(76) Inventor: David M. Duffy, Torrance, CA (US)

Correspondence Address: Bradley J. Bereznak, Burgess & Bereznak, LLP, Suite 180, 800 West El Camino Real, Mountain View, CA, 94040, US

(21) Appl. No.: 10/043,628

(22) Filed: Jan. 8, 2002

(51) Int. Cl. A61M 35/00

(52) U.S. Cl. 604/289

(57) ABSTRACT

According to the method of the present invention, a regulated vacuum pressure is applied to a skin defect (e.g., a depressed scar or wrinkle) on the surface of the skin. The vacuum pressure is either applied as a single treatment in preparation for other techniques, or as a structured series of applications to effect changes in the skin. The tissue is lifted using an air pressure differential, as may be generated by the vacuum source. It is emphasized that this abstract is provided to comply with the rules requiring an abstract that will allow a searcher or other reader to quickly ascertain the subject matter of the technical disclosure. It is submitted with the understanding that it will not be used to interpret or limit the scope or meaning of the claims. 37 CFR 1.72(b).

### FIELD OF THE INVENTION

The present invention relates generally to the repair of skin and soft tissue defects, including scars and wrinkles.

### BACKGROUND

Skin defects such as scarring from acne vulgaris, rhytids, stretch marks, wrinkles, and other types of traumatic and non-traumatic cutaneous depressions are difficult for physicians to treat. Although there are a number of treatment options available, most of these options are either not completely effective or produce unwanted side effects.

For example, dermal and subdermal fillers have been used for many years to improve the appearance of acne scar depressions and rhytids. Implants of inert biomaterials injected beneath an acne scar, for instance, build up the tissue under the scar, thereby reducing the depression caused by the scar. The injection of material into the body (and especially into the face) to obtain a more aesthetic appearance dates back to the turn of the nineteenth century. In the years prior to World War I, the injection of paraffin was used to correct facial contour defects. However, complications from this practice and the inability to achieve satisfactory long-terms results caused the procedure to be abandoned.

More recently, liquid silicone, liquid bovine collagen, fat, and other fibrin compounds have been used to clinically replace lost tissue volume by subcutaneous injection. Collagen suspensions such as liquid bovine collagen disclosed in U.S. Pat. Nos. 4,424,208, 4,582,640, and 4,642,117 are problematic in that they routinely fail to add endogeneous collagen to tissue. Although methods of recollagenation have been disclosed, such as the method discussed in U.S. Pat. No. 5,397,352, these solutions are also unsatisfactory. Because depressed scars may be hard and inelastic, they are sometimes unable to stretch and accommodate bio-fillers. In this setting, inert biomaterials are often dispersed to the border of the scar leaving the central area depressed or only slightly elevated. In general, bio-fillers work mechanically to elevate depressed contours and have no effect upon the intrinsic flexibility of the scar, possibly because they do not promote vascularization or molecular changes in scar tissue.

Lasers, dermabrasion, and chemical peeling have also been used to help stimulate the in-growth of collagen from other areas, thereby plumping unwanted facial contours. For example, laser dermal implants have been used for the treatment of facial skin depressions, such as described in U.S. Pat. No. 5,817,090. However, these techniques have been unable to safely treat the nadir of deep acne scars and wrinkles.

A new technique called “subscision” has recently been used to raise the base of a defect to the level of the surrounding skin. An example of this technique is disclosed in an article by Dr. David S. Orentreich and Dr. Norman Orentreich entitled, “Subcutaneous Incisionless (Subcision) Surgery for the Correction of Depressed Scars and Wrinkles,” published in Dermatol. Surg. (1995). The method involves a form of incisionless local subcuticular undermining where a tri-beveled hypodermic needle is inserted under the skin through a needle puncture to cut under a depressed scar, wrinkle, or contour. The skin is elevated by the act of surgically releasing the skin from its attachment to deeper tissues.

One shortcoming with the subscision technique is that because scar tissue is fibrotic, composed mostly of disorganized collagen fibrils that are stiff and bound down, it may be difficult to undermine the defect to get under the skin and create a pocket in which the anchoring fibers which hold the scar down are released permitting resumption of normal contours.

Therefore, what is needed is a new method and/or apparatus useful in treating scars and wrinkles either temporarily, by rendering them more amenable to the treatments described above, or permanently, by changing the tissue on a histological level.

### BRIEF DESCRIPTION OF THE DRAWINGS

The present invention will be understood more fully from the detailed description that follows and from the accompanying drawings, which however, should not be taken to limit the invention to the specific embodiments shown, but are for explanation and understanding only.

FIG. 1 is a schematic view of one embodiment of the present invention.

FIG. 2 illustrates an applicator used in one embodiment of the present invention.

FIG. 3A is a side view of an acne scar.

FIG. 3B shows the acne scar of FIG. 3A after being treated in accordance with one embodiment of the present invention.

FIG. 4A is a side view of a hypertrophic scar formation.

FIG. 4B shows the hypertrophic scar formation of FIG. 4A after treatment in accordance with another embodiment of the present invention.

FIG. 5 shows a step in a method for treatment of bound-down scars according to yet another embodiment of the present invention.

### DETAILED DESCRIPTION

A method and apparatus for the treatment of scar tissue and wrinkles is disclosed. In the following description numerous specific details are set forth, such as the particular configuration of mechanical devices, procedures, skin tissue types, etc., in order to provide a thorough understanding of the present invention. However, persons having ordinary skill in the medical arts will appreciate that these specific details may not be needed to practice the present invention.

According to the method of the present invention, a controlled vacuum is applied to a skin defect (i.e., a scar, wrinkle or damaged skin tissue) on the surface of the skin. The vacuum pressure is either applied as a single treatment in preparation for other techniques, or as one of a structured series of applications to permanently effect changes in the skin. The depressed tissue is lifted using an air pressure differential, as may be generated from a vacuum source, e.g., a vacuum pump. It should be understood, however, the vacuum pump described herein represents only one of a number of different possible embodiments of the present invention.

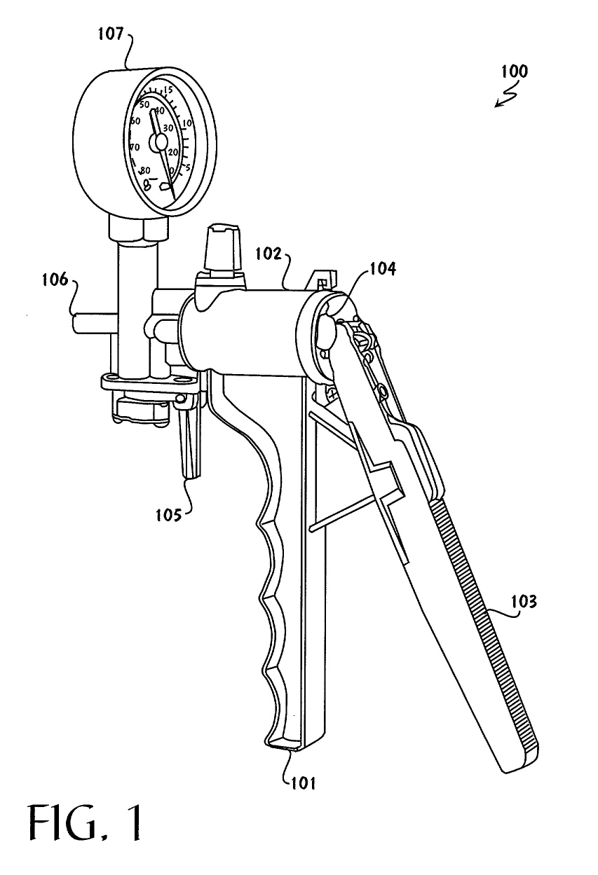

Referring now to FIG. 1 there is shown a schematic view of a vacuum pump device 100 that is exemplary of the type of pump that can be used in practicing the invention according to one embodiment. Although the vacuum pump device 100 illustrated by FIG. 1 is a hand-held mechanical pump, it is appreciated that a variety of different devices that provide a vacuum source may be utilized. For instance, in another embodiment the vacuum device may be electrically motorized and of an entirely different shape and size.

Vacuum pump device 100 provides a controlled vacuum pressure for the elevation and/or remodeling of depressed scar tissue and wrinkles. The hand-actuated pump of FIG. 1 allows for operator control. In an exemplary embodiment, vacuum pump device 100 comprises a hand-held device called the Mityvac® vacuum pump, manufactured by Prism Enterprises of San Antonio, Tex.

In the embodiment illustrated by FIG. 1, the body of pump device 100 comprises a fixed handle 101 attached to a sealed cylinder 102. A movable handle 103 is pivotally attached at a joint 104 affixed to handle 101. One portion or end of movable handle 103 is mechanically coupled via joint 104 to a piston rod (not shown in this view). In operation, the pair of handles 101 and 103 may be gripped between the thumb and forefingers, and then squeezed so that movable handle 103 draws closer to fixed handle 101. This causes the piston rod to be drawn back, resulting in the creation of a vacuum pressure at the vacuum fitting. When the pair of handles 101 and 103 is released, a spring mechanism causes the piston to return to the inner end of the cylinder 102.

There are a variety of basic techniques that may be utilized to release vacuum at the pump and these are well known in the art. For example, in the apparatus shown in FIG. 1 a vacuum release trigger 105 is used. Trigger 105 is a straight lever, which, when pulled straight back, functions to vent air into the system, thus relieving the vacuum pressure generated at the vacuum fitting.

Flexible tubing (not shown in this view) may attach to the vacuum port 106 on one end, and to an applicator tip (not shown in this view) on the other end. The vacuum pressure created at the vacuum source is applied to the applicator tip through the tubing. In this mechanism, the applicator tip is coordinated with the size and shape of the skin tissue to be elevated and/or remodeled. In other words, a great variety of different shaped applicator tips, each having a size and shape configured to fit over an equally different variety of depressed skin tissues, may be utilized. In one embodiment, an appropriately sized and shaped applicator tip is selected and attached to the applicator tip.

A vacuum gauge and/or regulator 107 may be used to regulate the applied vacuum pressure. Regulation of vacuum pressure prevents contusions or other damage to the scar tissue or wrinkles that might result if the negative pressure applied through the applicator tip is too strong. A timed regulator may also be used to guard against applications that exceed a predetermined duration or are otherwise too prolonged.

In one embodiment, a constant vacuum pressure in the range of 10-80″ Hg may be used repetitively (e.g., 3-4 times) for predetermined time intervals (e.g., 5-10 seconds each time) to elevate an ordinary wrinkle located near the corner of a human eye. The specific vacuum pressure, number of times the vacuum is applied, and the length of the application intervals may vary widely based on individual patient response to the procedure and also on the nature, location, and type of skin defect (e.g., an acne scar, keloid scar, a wrinkle, etc.) being treated, as well as the particular result to be achieved (e.g., temporary or permanent restoration).

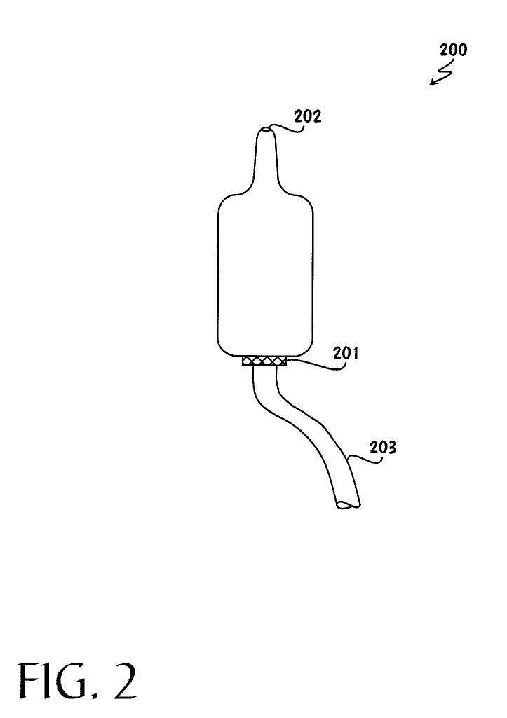

Referring now to FIG. 2 there is shown an example of a customized applicator 200 used in connection with a vacuum device according to one embodiment of the present invention. The applicator 200 may be removeably attached to the discharge end of tubing 203 by way of an attachment means that provides for an air-tight seal 201 at a prescribed range of vacuum pressure. The air-tight seal 201 is made of a flexible material which is resilient and possesses some expandable/compressible characteristics. A tip 202 of applicator 200 is designed to completely encompass a depressed skin defect (e.g., an acne scar, a wrinkle, a keloid scar, etc.). The tip 202 has a size and shape that matches the shape and size of the skin defect, fitting on the outside edge of the surface of the skin depression that is to be elevated and/or remodeled by the method disclosed herein.

As is depicted in FIG. 2, the portion (i.e., the tip 202) of the applicator 200 that encompasses the skin defect is designed to match the shape and size of the scar and/or wrinkle that is to be treated. Thus, because the applicator tip 202 shown in FIG. 2 is designed to treat an acne scar, the tip 202 matches the size (i.e., the circumference) of the acne scar on the surface of a patient's skin (not shown in this view). The shape and size of the applicator tip 202 may therefore vary depending on the shape and size of the skin defect to be treated. An applicator tip 202 designed to treat a linear scar on a patient's cheek, for instance, would be shaped to match the length and width of the linear scar.

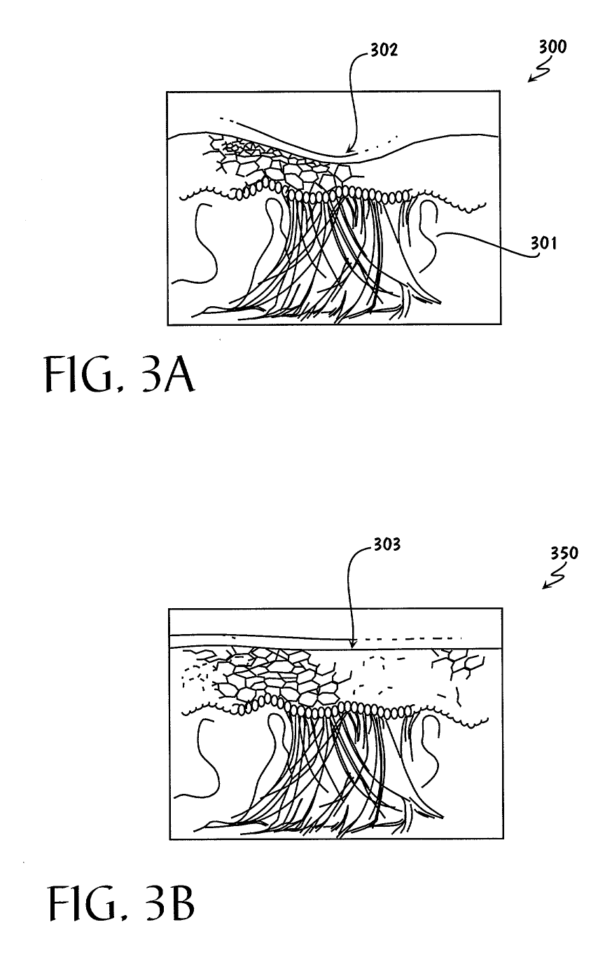

Referring now to FIG. 3A, there is shown a side view of an acne scar 300 before being treated with a vacuum device. Fibrous bands of tissue 301 tether the scar surface down, forming a depression 302 on the surface of the skin. FIG. 3B is a side view of the acne scar of FIG. 3A after the scar has been treated with a vacuum device 350 according to one embodiment of the present invention. In order to achieve the results illustrated by FIG. 3B, the applicator tip (not shown in this view) is placed over the acne scar.

As discussed above, in one embodiment tubing may be connected at one end to an applicator. The other end of the tubing may be connected to a vacuum pump. After the tip is placed on the patient's skin in the area of treatment, the pump is turned on. The vacuum pressure is applied for a length of time sufficient to raise the surface 303 of the scar tissue to the same level as the surface of the surrounding skin, thus creating the restored topography illustrated by FIG. 3B. Multiple applications (i.e., an appropriate regimen of vacuum and rest) may be required over a period of days or even weeks, depending upon how fast the scar tissue responds to the treatment. For instance, a vacuum pressure of 10-80″ Hg applied to a small acne scar of a circumference of 2 mm. repeated daily (e.g., on an hourly basis) for 30-second intervals over a period of weeks may affect changes on a histological level in the skin tissue, thus effecting a permanent change in the topography of the skin and creating a more aesthetic appearance.

The scar tissue may be elevated/remodeled through the use of the vacuum alone. When adequately stressed in this manner, human cells increase in size and external structure and also deviate to accommodate the vacuum or negative force that is applied to the cell. Therefore, application of the vacuum device to the acne scar of FIG. 3A over a period of weeks or months (depending on a patient's individual response to therapy) may eventually induce desirable histological and clinical changes in the texture and appearance of scars and wrinkles as tissue remodels to accommodate vacuum induced stresses.

The present invention may also be used in combination with a variety of other treatments for depressed skin defects (e.g., distensile scars, bound-down scars, skin grafts, wrinkles, depressed contours, cellulite dimples, etc.). As the vacuum pump device lifts and expands depressed skin tissue a variety of filling materials may be injected in or under the tissues to effect desired contour changes. Alternatively, bioactive materials may be injected into or under the depressed skin tissue following treatment with the vacuum pump device described above.

By way of example, FIG. 3A illustrates an acne scar bound down by fibrous scar tissue 301. Such fibrous scar tissue makes it difficult for a physician to effectively create a pocket underneath the skin surface for which to inject the materials that are clinically used to replace lost tissue volume. By applying the vacuum pressure of the present invention, the physician may be able to more readily perform inter or sub-lesional injections beneath depressed scars or wrinkles, because the tissue has been stretched by the applied vacuum, rendering it more elastic and more easily elevated.

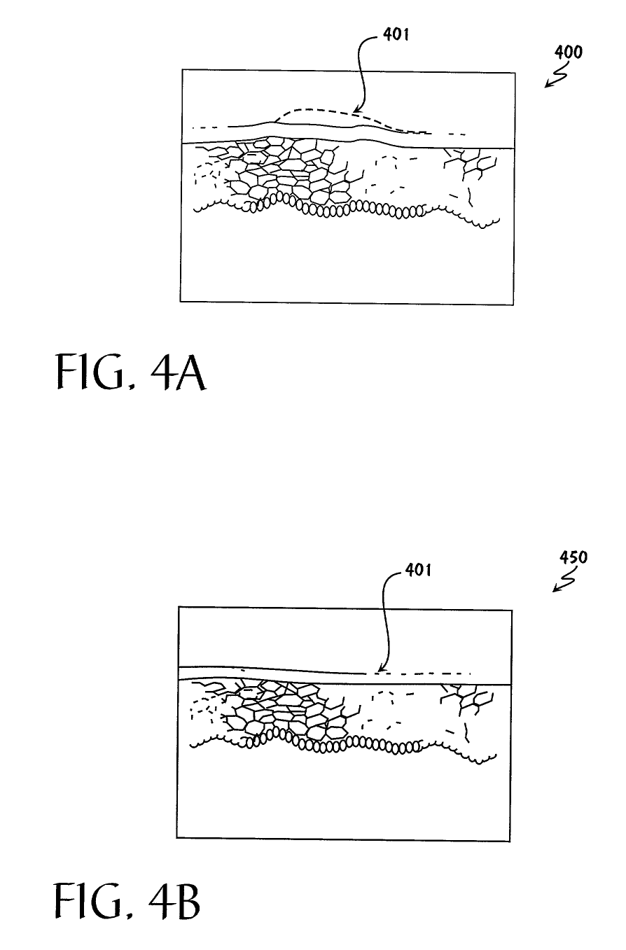

The present invention may also be used upon hypertrophic or keiloidal elevated scars. FIG. 4A is a side view of a hypertrophic scar formation before being treated with the vacuum device and a cortisone injection 400. The hypertrophic scar 401 is a raised fibrotic projection of the skin. These types of scars often occur after burn injuries and as a consequence of wounds in a specific area (i.e., sternum and shoulder girdle, as well as occurring in certain skin types which are prone to this problem). Typically hypertrophic scars are treated with a variety of modalities including x-rays, oral and injectable cortisone which modify their structure through as yet unknown mechanisms. In accordance with this particular embodiment of the present invention, the hypertrophic scar formation of FIG. 4A is treated by applying a vacuum pressure to the afflicted skin tissue as described previously. In conjunction with, or immediately following, the application of vacuum pressure, cortisone is injected into the scar tissue.

FIG. 4B is a side view of the hypertrophic scar of FIG. 4A following treatment with the vacuum device and injection of cortisone 450.

It should be understood that within the context of the present application, the term “injected into” means injecting the cortisone or other agents either into the scar or proximate to the scar tissue. In this instance, the vacuum device is first used on the hypertrophic scar 401 according to the method described herein. Methodically subjecting the scar tissue to vacuum treatment may change the appearance and structural characteristics of the scar. Vacuum may create a more flexible scar, which is easier to inject using cortisone resulting in a more ameliorization of the scar itself or decrease in its contractual qualities. Thus, the hypertrophic scar 401 is smoother after the treatment.

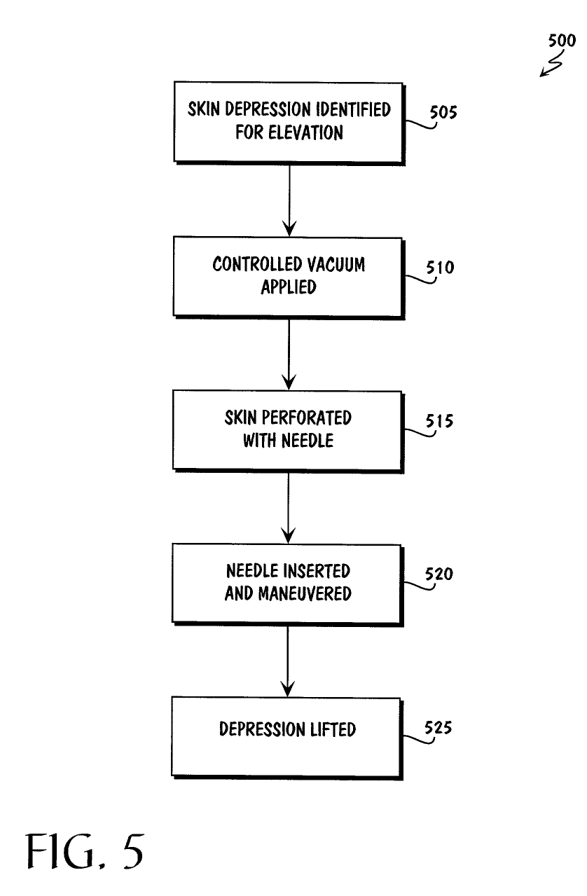

Referring now to FIG. 5 there is shown a view of the steps in a method for treatment of bound-down scars using a vacuum device followed by subscision 500 according to another embodiment of the present invention. First, the physician identifies a skin depression for elevation (block 505). A controlled vacuum is then applied in a pre-determined program protocol, either with repeated applications pretreatment, or 5 to 10 minutes at the time of treatment sufficient to raise the surface of the scar or create a cleavage line at the right level for incision of the hypodermic needle (block 510). The skin is perforated with the desired instrument adjacent to the skin depression (block 515). A tri-beveled hypodermic needle or other instrument is inserted through the perforation and its sharp edges are maneuvered under the defect to make subcuticular cuts (block 520). The depression is lifted by the releasing action of the procedure, as well as from connective tissue that forms in the course of normal wound healing (block 525). Vacuuming will also be of value in enhancing the elevation of the skin in a specific protocol after subscisional surgery.

The method of the present invention also works well as an adjunct to a variety of other skin resurfacing methods, including dermabrasion, laser therapy, and chemical peels.

In the foregoing, apparatus and methods have been described for treatment of skin defects. Although the present invention has been described with reference to specific exemplary embodiments, it should be understood that numerous changes in the disclosed embodiments may be made in accordance with the disclosure herein without departing from the spirit and scope of the invention. The preceding description, therefore, is not meant to limit the scope of the invention. Rather, the scope of the invention is to be determined only by the appended claims and their equivalents

I claim:

1. A method for treating a depressed skin defect, comprising:
(a) applying a vacuum pressure to the depressed skin defect;
(b) regulating the vacuum pressure within a prescribed range of pressures; and
(c) releasing the vacuum pressure after a time interval.

2. The method of claim 1 further comprising repeating (a)-(c) one or more times.

3. The method of claim 1 wherein the vacuum pressure is regulated at or above 10 inches Hg and at or below 80 inches Hg.

4. The method of claim 1 wherein the vacuum pressure is regulated at a constant pressure level.

5. The method of claim 1 wherein the depressed skin defect is a scar.

6. The method of claim 1 wherein the depressed skin defect is a wrinkle.

7. The method of claim 1 further comprising injecting a bioactive material into the depressed skin defect.

8. The method of claim 1 further comprising performing subcision on the depressed skin defect.

9. The method of claim 1 further comprising performing laser therapy on the depressed skin defect.

10. The method of claim 1 further comprising performing chemical peeling, dermabrasion, or other skin resurfacing treatments on the depressed skin defect.

11. The method of claim 1 wherein the vacuum pressure is regulated at a pressure sufficient to elevate the depressed skin defect, but less than a pressure that causes damage to tissue surrounding the depressed skin defect.

12. An apparatus for treating a depressed area of skin tissue comprising:
a vacuum source to generate a vacuum pressure;
an applicator having a first end coupled to the vacuum source and a second end configured with an opening, the depressed area of skin tissue being subjected to the vacuum pressure upon placement of the opening on the depressed area of skin tissue.

13. The apparatus of claim 12 wherein the opening comprises an applicator tip configured to surround the depressed area of skin tissue.

14. The apparatus of claim 12 wherein the vacuum pressure is generated by a vacuum pump.

15. The apparatus of claim 12 wherein the vacuum pressure is regulated within prescribed range of pressures.

16. The apparatus of claim 15 wherein the vacuum pressure is regulated at or above 10 inches Hg and at or below 80 inches Hg.

17. The apparatus of claim 12 wherein the vacuum pressure is released after a time interval.

18. The apparatus of claim 12 wherein the applicator may be removeably attached to the vacuum source.

19. The apparatus of claim 12 wherein the applicator is attached to the vacuum source by an air-tight seal at a prescribed range of vacuum pressure.

20. The apparatus of claim 12 wherein the opening of the applicator is designed to match the shape and size of the depressed area of skin tissue to be treated.

21. The apparatus of claim 12 wherein the depressed area of skin tissue to be treated is a scar.

22. The apparatus of claim 12 wherein the depressed area of skin tissue to be treated is a wrinkle.

23. The apparatus of claim 12 wherein the apparatus is used as an adjunct to injection of bioactive material into the depressed area of skin tissue.

24. The apparatus of claim 12 wherein the apparatus is used as an adjunct to subcision on the depressed area of skin tissue.

25. The apparatus of claim 12 wherein the apparatus is used as an adjunct to laser therapy on the depressed area of skin tissue.

26. The apparatus of claim 12 wherein the apparatus is used as an adjunct to chemical peeling, dermabrasion, or other skin resurfacing treatments.

27. A method for treating a depressed skin defect, comprising:
applying a vacuum pressure to the depressed skin defect;
regulating the vacuum pressure within a prescribed range of pressures;
releasing the vacuum pressure after a time interval; and
injecting a bioactive material into the depressed skin defect.

28. The method of claim 27 wherein the bioactive material is liquid silicone.

29. The method of claim 27 wherein the bioactive material is collagen.

30. The method of claim 27 wherein the vacuum pressure is regulated at or above 10 inches Hg and at or below 80 inches Hg.

31. The method of claim 27 wherein the vacuum pressure is regulated at a constant pressure level.

32. The method of claim 27 wherein the depressed skin defect is a scar.

33. The method of claim 27 wherein the depressed skin defect is a wrinkle.

34. The method of claim 27 wherein the vacuum pressure is regulated at a pressure sufficient to elevate the skin defect, but less than a pressure that causes damage to tissue surrounding the skin defect.

35. A method for treating a depressed skin defect, comprising:
applying a vacuum pressure to the depressed skin defect;
regulating the vacuum pressure within a prescribed range of pressures;
releasing the vacuum pressure after a time interval; and
performing subcision on the depressed skin defect.

36. The method of claim 35 wherein the vacuum pressure is regulated at or above 10 inches Hg and at or below 80 inches Hg.

37. The method of claim 35 wherein the vacuum pressure is regulated at a constant pressure level.

38. The method of claim 35 wherein the depressed skin defect is a scar.

39. The method of claim 35 wherein the depressed skin defect is a wrinkle.

40. The method of claim 35 wherein the vacuum pressure is regulated at a pressure sufficient to elevate the skin defect, but less than a pressure that causes damage to tissue surrounding the skin defect.

41. A method for treating a depressed skin defect, comprising:
applying a vacuum pressure to the depressed skin defect;
regulating the vacuum pressure within a prescribed range of pressures;
releasing the vacuum pressure after a time interval; and
performing laser therapy on the depressed skin defect.

42. The method of claim 41 wherein the vacuum pressure is regulated at or above 10 inches Hg and at or below 80 inches Hg.

43. The method of claim 41 wherein the vacuum pressure is regulated at a constant pressure level.

44. The method of claim 41 wherein the depressed skin defect is a scar.

45. The method of claim 41 wherein the depressed skin defect is a wrinkle.

46. The method of claim 41 wherein the vacuum pressure is regulated at a pressure sufficient to elevate the skin defect, but less than a pressure that causes damage to tissue surrounding the skin defect.

47. A method for treating a depressed skin defect, comprising:
applying a vacuum pressure to the depressed skin defect;
regulating the vacuum pressure within a prescribed range of pressures;
releasing the vacuum pressure after a time interval; and
performing chemical peeling, dermabrasion, or other skin resurfacing treatments on the depressed skin defect.

48. The method of claim 47 wherein the vacuum pressure is regulated at or above 10 inches Hg and at or below 80 inches Hg.

49. The method of claim 47 wherein the vacuum pressure is regulated at a constant pressure level.

50. The method of claim 47 wherein the depressed skin defect is a scar.

51. The method of claim 47 wherein the depressed skin defect is a wrinkle.

52. The method of claim 47 wherein the vacuum pressure is regulated at a pressure sufficient to elevate the skin defect, but less than a pressure that causes damage to tissue surrounding the skin defect.

53. A method for treating a hypertrophic scar, comprising:
(a) applying a vacuum pressure to the hypertrophic scar;
(b) regulating the vacuum pressure within a prescribed range of pressures; and
(c) releasing the vacuum pressure after a time interval.

54. The method of claim 53 further comprising repeating (a)-(c) one or more times.

55. The method of claim 53 wherein the vacuum pressure is regulated at or above 10 inches Hg and at or below 80 inches Hg.

56. The method of claim 53 wherein the vacuum pressure is regulated at a constant pressure level.

57. The method of claim 53 further comprising injecting a skin-weakening medicament into the hypertrophic scar.

58. The method of claim 57 wherein the skin-weakening medicament is cortisone.

59. The method of claim 53 wherein the vacuum pressure is regulated at a pressure sufficient to soften the hypertrophic scar, but less than a pressure that causes damage to tissue surrounding the hypertrophic scar.

60. An apparatus for treating a hypertrophic scar comprising:
a vacuum source to generate a vacuum pressure; and
an applicator having a first end coupled to the vacuum source and a second end configured with an opening, the hypertrophic scar being subjected to the vacuum pressure upon placement of the opening on the hypertrophic scar.

61. The apparatus of claim 60 wherein the opening comprises an applicator tip configured to surround the hypertrophic scar.

62. The apparatus of claim 60 wherein the vacuum pressure is generated by a vacuum pump.

63. The apparatus of claim 60 wherein the vacuum pressure is regulated within a prescribed range of pressures.

64. The apparatus of claim 63 wherein the vacuum pressure is regulated at or above 10 inches Hg and at or below 80 inches Hg.

65. The apparatus of claim 60 wherein the vacuum pressure is released after a time interval.

66. The apparatus of claim 60 wherein the applicator may be removeably attached to the vacuum source.

67. The apparatus of claim 60 wherein the applicator is attached to the vacuum source by an air-tight seal at a prescribed range of vacuum pressure.

68. The apparatus of claim 60 wherein the opening of the applicator is designed to match the shape and size of the hypertrophic scar.

69. The apparatus of claim 60 wherein the apparatus is used as an adjunct to injection of a skin-weakening medicament into the hypertrophic scar.

70. The apparatus of claim 69 wherein the skin-weakening medicament is cortisone.
